(19) United States
(12) Patent Application Publication
(10) Pub. No.: US 2023/0049330 A1
(43) Pub. Date: Feb. 16, 2023

(54) USE OF QX314 TO PREVENT SYMPATHOEXCITATION ASSOCIATED WITH ADMINISTRATION OF TRPV1 MODULATORS

(71) Applicant: NEUCURES, INC., Rolling Hills, CA (US)

(72) Inventor: Pradeep S. Rajendran, Los Angeles, CA (US)

(21) Appl. No.: 17/909,271

(22) PCT Filed: Mar. 3, 2021
(86) PCT No.: PCT/US2021/020574

§ 371 Date: Sep. 2, 2022

Related U.S. Application Data
(60) Provisional application No. 62/985,017, filed on Mar. 4, 2020.

(51) Int. Cl. A61K 31/165; A61K 31/05; A61K 31/26

(52) U.S. Cl. CPC A61K 31/165; A61K 31/05; A61K 31/26

(57) ABSTRACT

Methods and compositions for preventing sympathetic activation resulting from administration of a TRPV1 modulator alone. Methods include co-administering QX-314 and a TRPV1 modulator to prevent sympathetic activation resulting from administration of a TRPV1 modulator alone comprising administering QX-314 and the TRPV1 modulator to a subject in need thereof. Compositions include a composition to prevent sympathetic activation comprising QX-314 and a TRPV1 modulator.

### CROSS-REFERENCE TO RELATED APPLICATION

This application claims priority to U.S. Provisional Application No. 62/985,017, filed Mar. 4, 2020, entitled, “USE OF QX314 TO PREVENT SYMPATHOEXCITATION ASSOCIATED WITH ADMINISTRATION OF TRPV1 MODULATORS”, which is incorporated by reference in its entirety herein.

### BACKGROUND

Transient receptor potential vanilloid (TRPV) channels are a family of nonselective cation channels that are present on the membranes of various cells throughout the body. Of this family, TRPV1 channels located on afferent neurons within the nervous system are responsible for the transduction of noxious stimuli. Exogenous modulators of TRPV1 such as capsaicin and resiniferatoxin (RTX) are agonists that depolarize TRPV1-expressing neurons and can be specifically used to temporarily or permanently desensitize this population of neurons. Thus, the use of exogenous TRPV1 modulators (e.g., capsaicin, RTX) are emerging as a treatment for a range of clinical conditions including pain, cardiovascular diseases, diabetes, asthma, cancer, arthritis, and cystitis, where deactivation of TRPV1-expressing neurons plays an important role.

During the pathogenesis of conditions such as cardiovascular diseases (e.g., hypertension, myocardial infarction (MI), arrhythmias, heart failure (HF)), diabetes, lung diseases, cancers, arthritis, and urinary tract diseases, activation of TRPV1 channels on afferent neurons by endogenous ligands leads to reflex activation of efferent neurons within the sympathetic nervous system (SNS). Acutely following cardiac injury such as MI, TRPV1-mediated sympathetic activation helps maintain cardiac output and preserve life-sustaining circulation. In addition, TRPV1 plays a role in sympathetic activation in conditions such as hypertension. However, chronic sympathetic activation leads to maladaptive remodeling of the heart and the nervous system, which contributes to the progression of cardiovascular diseases. Epicardial and thoracic epidural administration of TRPV1 modulators have been demonstrated to inhibit afferent nerve function, which decreases fibrosis and arrhythmogenesis following MI and slows the progression of HF.

However, administration of TRPV1 modulators induces a transient sympathoexcitation, observed as a rise in heart rate and blood pressure. In most patients, this transient increase is tolerated; however, in some patients, particularly those with already compromised cardiac function (e.g., MI, HF), this can lead to adverse cardiac events such as arrhythmias and even death. Such side effects severely limit the utility of potent TRPV1 modulators as therapeutic agents for not only cardiovascular diseases but also other conditions. As such, there is need for a novel method of administering TRPV1 modulators that limits the risk of sympathoexcitation while still preserving their ability to inhibit afferent nerve function.

QX-314 (N-ethyl-lidocaine) is a cationic lidocaine analog that blocks voltage-dependent sodium channels. It is membrane impermeable and requires membrane translocation to bind the intracellular portion of the sodium channel, where it exerts its sodium channel blockade.

### SUMMARY

The disclosed subject matter provides a method of co-administering QX-314 and a TRPV1 modulator to prevent sympathetic activation resulting from administration of a TRPV1 modulator alone comprising administering QX-314 and the TRPV1 modulator to a subject in need thereof.

In one embodiment, QX-314 is administered before the TRPV1 modulator. In another embodiment, QX-314 and the TRPV1 modulator are administered at the same time. In any of the above embodiments, administering QX-314 or the TRPV1 modulator comprises administration by at least one of topical, subcutaneous, epicardial, epidural, intrathecal, peri or intra-ganglionic, vascular, intraarticular, interarticular, pericardial, intrapericardial, or intravesical administration. In another embodiment, QX-314 and the TRPV1 modulator are administered by the same method of administration. In another embodiment, QX-314 and the TRPV1 modulator are administered by different methods of administration. In any of the above embodiments, the TRPV1 modulator is selected from the group consisting of capsaicin, nordihydrocapsaicin, dihydrocapsaicin, homodihydrocapsaicin, homocapsaicin, nonivamide, bradykinin, RTX, Iodo-RTX, tinyatoxin, allyl isothiocyanate, N- arachidonoylaminophenol, N-Vanillylarachidonamide, N-oleoyl-dopamine, olvanil, palvanil, cannabidiol, capsiate, capsaicin O-ethyl (trimethylammonium) acetate, capsaicin O-butyl(trimethylammonium) acetate, capsaicin O-tetraethylammonium acetate, zucapsaicin, or change in temperature or pH. In any of the above embodiments, QX-314 is administered to the subject at a concentration of about 1 mM to about 100 mM. By way of example but not limitation, QX-314 may be administered to the subject at a concentration of about 10 mM to about 40 mM, about 10 mM, about 20 mM, or about 40 mM. In any of the above embodiments, the TRPV1 modulator is administered to the subject at a concentration of about 0.1 µg/ml to about 125 µg/ml. By way of example but not limitation, the TRPV1 modulator may be administered to the subject at a concentration of about 2.5 µg/ml to about 12.5 µg/ml or about 12.5 µg/ml. In any of the above embodiments, the administration of QX-314 and the TRPV1 modulator results in preventing sympathetic activation for about 0 to about 60 minutes following the step of administering QX-314 and the TRPV1 modulator that would result if the subject had been administered with TRPV1 modulator alone. In any of the above embodiments, the administration of QX-314 and the TRPV1 modulator results in the subject having lower heart rate or blood pressure for about 0 to about 60 minutes following the step of administering QX-314 and the TRPV1 modulator than if the subject had been administered with TRPV1 modulator alone.

The disclosed subject matter also provides a composition to prevent sympathetic activation comprising QX-314 and a TRPV1 modulator.

In one embodiment, the composition comprises a mixture of QX-314 and the TRPV1 modulator. In any of the above embodiments, the TRPV1 modulator is selected from the group consisting of capsaicin, nordihydrocapsaicin, dihydrocapsaicin, homodihydrocapsaicin, homocapsaicin, nonivamide, bradykinin, RTX, Iodo-RTX, tinyatoxin, allyl isothiocyanate, N-arachidonoylaminophenol, N-Vanillylarachidonamide, N-oleoyl-dopamine, olvanil, palvanil, cannabidiol, capsiate, capsaicin O-ethyl (trimethylammonium) acetate, capsaicin O-butyl(trimethylammonium) acetate, capsaicin O-tetraethylammonium acetate, zucapsaicin, or a compound that changes temperature or pH. In any of the above embodiments, QX-314 comprises a concentration of about 1 mM to about 100 mM. By way of example but not limitation, QX-314 may comprise a concentration of about 10 mM to about 40 mM, about 10 mM, about 20 mM, or about 40 mM. In any of the above embodiments, the TRPV1 modulator comprises a concentration of about 0.1 µg/ml to about 125 µg/ml. By way of example but not limitation, the TRPV1 modulator may comprise a concentration of about 2.5 µg/ml to about 12.5 µg/ml or about 12.5 µg/ml. In any of the above embodiments, the composition further comprises at least one excipient. In one embodiment, the excipient is selected from at least one of the group consisting of ethanol, methanol, polyethylene glycol, dimethyl sulfoxide, sodium chloride, or a cyclodextran.

### DRAWINGS

FIG. 1 illustrates the intrapericardial administration of a mixture of TRPV1 and QX-314.

FIGS. 2A-C show effects of intrapericardial administration of RTX versus RTX + QX-314 on heart rate and blood pressure.

### DETAILED DESCRIPTION

The present disclosure provides compositions of TRPV1 modulators with QX-314; and methods for co-administration of TRPV1 modulators with QX-314. In methods of the present disclosure, co-administration of QX-314 with TRPV1 modulators mitigates transient TRPV1-mediated sympathoexcitation, and thereby, limits the risk of adverse cardiovascular events.

As used herein, the singular forms “a”, “an” and “the” include plural referents unless the context clearly dictates otherwise. The use of the term “or” in the claims and the present disclosure is used to mean “and/or” unless explicitly indicated to refer to alternatives only or the alternatives are mutually exclusive.

As used herein, the term “TRPV1 modulator” is meant to include anything that modulates TRPV1 activity. By way of example but not limitation, such TRPV1 modulators may include compounds such as capsaicin, nordihydrocapsaicin, dihydrocapsaicin, homodihydrocapsaicin, homocapsaicin, nonivamide, bradykinin, RTX, Iodo-RTX, tinyatoxin, allyl isothiocyanate, N-arachidonoylaminophenol, N-Vanillylarachidonamide, N-oleoyl-dopamine, olvanil, palvanil, cannabidiol, capsiate, capsaicin O-ethyl (trimethylammonium) acetate, capsaicin O-butyl(trimethylammonium) acetate, capsaicin O-tetraethylammonium acetate, or zucapsaicin. By way of example but not limitation, TRPV1 modulator may comprise:

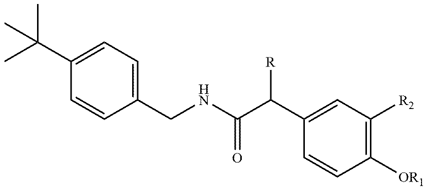

wherein R is H or CH₃; R₁ is H or CH₂CH₂NH₂; and R₂ is OCH₃, F, or Cl.

By way of example but not limitation, the TRPV1 modulator may comprise:

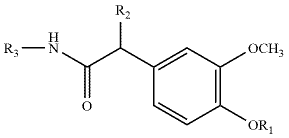

wherein R₁ is H; R₂ is H or CH₃; and R₃ is (CH₂)₇CH₃, (CH₂)₈CH₃, or (CH₂)₃Ph(3,4-Me₂); or wherein R1 is CH₂CH₂NH₂; R₂ is H or CH₃; and R₃ is (CH₂)₃Ph(3,4-Me₂).

By way of example but not limitation, the TRPV1 modulator may comprise:

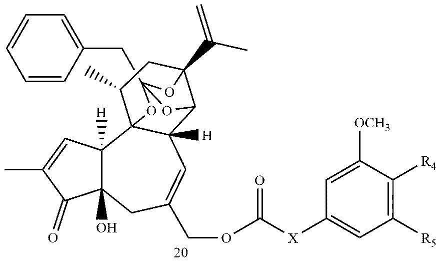

wherein X is CH₂ or O; R₄ is OH; and R₅ is H or I; or wherein X is CH₂; R₄ is NH₂ or NHSO₂CH₃; and R₅ is H.

By way of example but not limitation, the TRPV1 modulator may comprise:

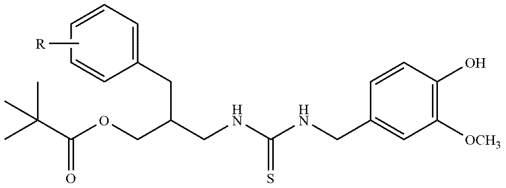

wherein R is 3,4-Me₂ or 4-tBu.

By way of example but not limitation, the TRPV1 modulator may comprise:

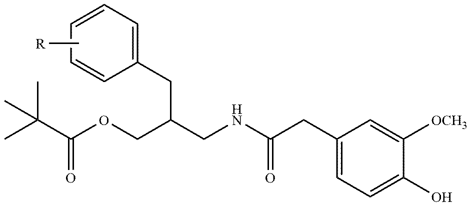

wherein R is 3,4-Me₂ or 4-tBu.

By way of example but not limitation, the TRPV1 modulator may comprise:

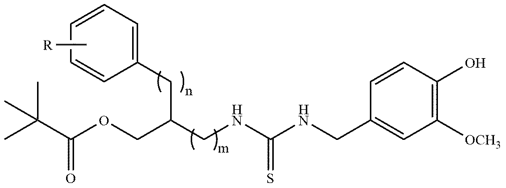

wherein m is 0 or 1; n is 1 or 2; and R is 3,4-Me₂ or t-Bu.

By way of example but not limitation, the TRPV1 modulator may comprise:

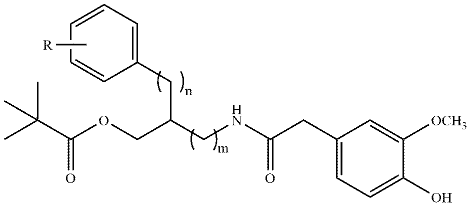

wherein m is 0 or 1; n is 1 or 2; and R is 3,4-Me₂ or t-Bu.

By way of example but not limitation, the TRPV1 modulator may comprise:

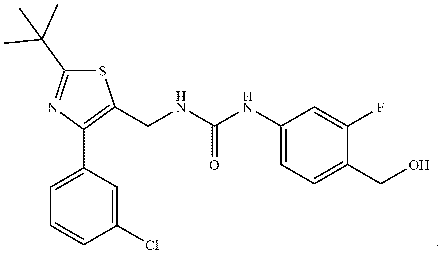

By way of example but not limitation, the TRPV1 modulator may comprise:

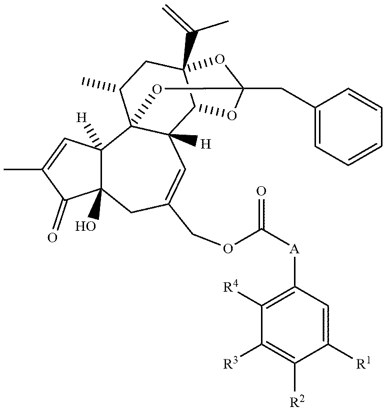

wherein A is nothing or —CH₂—CH₂—; R¹ is OMe; R² is OH; R³ is H or I; and R⁴ is H or I; or wherein A is nothing, —CH₂, —CH₂—CH₂—, or (E) —CH═CH—; R¹, R², R³, and R⁴ are H; or wherein A is nothing, —CH₂—CH₂—, or (E) —CH═CH—; R¹ is OMe, R² is OH, R³ is H, and R⁴ is H.

By way of example but not limitation, the TRPV1 modulator may comprise:

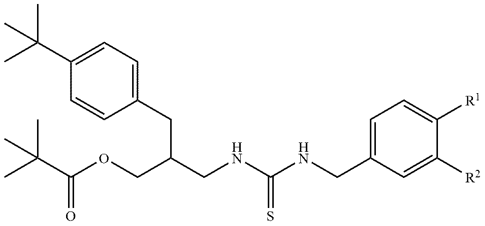

wherein R¹ is OH and R² is OCH₃; or wherein R¹ is NHSO₂CH₃ and R² is F.

By way of example but not limitation, TRPV1 modulators may also include compounds or environmental factors that result in changes in temperature and pH.

As used herein, the term “QX-314” or “QX-314 bromide” is meant to include N-Ethyl lidocaine bromide or other lidocaine analogs, or other sodium channel blockers that act on the intracellular domains.

The present disclosure provides methods for co-administration of TRPV1 modulators with QX-314.

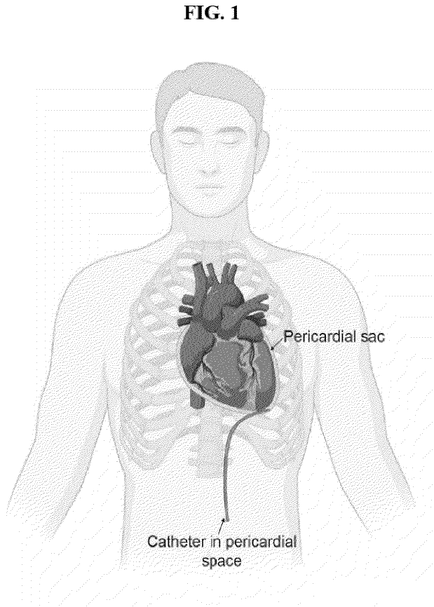

Administration of TRPV1 modulators and QX-314 can be performed by methods known in the art, including but not limited to topical, subcutaneous, epicardial, epidural, intrathecal, peri or intra-ganglionic, vascular, intraarticular, interarticular, pericardial, intrapericardial, and intravesical routes of administration. In some embodiments, the TRPV1 modulator and QX-314 are administered via the same route. In some embodiments, the TRPV1 modulator and QX-314 are administered via different routes. By way of example but not limitation, QX-314 may be administered via IV infusion while the TRPV1 modulator is delivered to the target tissue by other means. By way of example but not limitation, the TRPV1 modulator and QX-314 may be administered by catheter inserted in the pericardial space as illustrated in FIG. 1.

Administration of the TRPV1 modulator and QX-314 can be sequential or simultaneous. In some embodiments, QX-314 is administered before the TRPV1 modulator. By way of example but not limitation, QX-314 is administered locally or intravenously minutes prior to local administration of the TRPV1 modulator. In another embodiment, QX-314 is injected locally and allowed to dissipate, following which the TRPV1 modulator is administered. In another embodiment, the TRPV1 modulator is administered as a small dose, followed by QX-314, and then a subsequent larger dose of the TRPV1 modulator is administered. In some embodiments, QX-314 is administered at the same time as TRPV1 modulators. By way of example but not limitation, simultaneous co-administration may comprise the TRPV1 modulator and QX-314 administered as a pre-mixed solution, or, one compound may be applied before the other is absorbed.

In some embodiments, the methods disclosed herein are used to treat acute or chronic cardiovascular disease, acute or chronic pain, acute or chronic lung disease such as chronic asthma or obstructive pulmonary disease, acute or chronic arthritis, acute or chronic radiculopathy, hypertension, myocardial infarction, arrhythmias, heart failure, or other conditions for which chronic inflammation is an important pathophysiologic factor and neural desensitization is desired.

The present disclosure provides compositions of a TRPV1 modulator and QX-314.

In some embodiments, the composition comprises a co-mixture of both a TRPV1 modulator and QX-314.

In some embodiments, the composition comprises a TRPV1 modulator and QX-314 that are mixed at administration. In some embodiments, mixing of the TRPV1 modulator and QX-314 may occur in the subject or before administration. By way of example but not limitation, in some embodiments QX-314 is administered to the subject and then the TRPV1 modulator is administered. In other embodiments, the TRPV1 modulator is mixed with QX-314 prior to administration.

In some embodiments, wherein the TRPV1 modulator is a compound, the TRPV1 modulator is administered at a concentration sufficient to modulate TRPV1 activity. By way of example but not limitation, such concentration of the TRPV1 modulator is about 0.1 µg/ml to 125 µg/ml. By way of example but not limitation, said concentration of the TRPV1 modulator is about 2.5 µg/ml to 12.5 µg/ml, about 2.5 µg/ml, about 5 µg/ml, about 7.5 µg/ml, about 10 µg/ml, or about 12.5 µg/ml.

In some embodiments, QX-314 is administered at concentration range of about 1 mM to 100 mM. By way of example but not limitation, in some embodiments, said concentration of QX-314 is from about 10 mM to about 40 mM, about 10 mM, about 20 mM, or about 40 mM.

In some embodiments, the composition further comprises one or more excipients. Such excipients include those known in the art, by way of example but not limitation, said excipients include bulking agents, fillers, or diluents. By way of example but not limitation, said excipients include ethanol, methanol, polyethylene glycol, tween, dimethyl sulfoxide (“DMSO”), sodium chloride, and cyclodextrans, or any other bulking agent, filler, or diluent.

### EXAMPLES

The following example is provided to better illustrate the methods of the present disclosure and the resultant effects on a chronic disease (specifically myocardial infarction). This example is not intended to be limited or to otherwise alter the scope of the methods and compositions disclosed in the present disclosure.

### Example 1

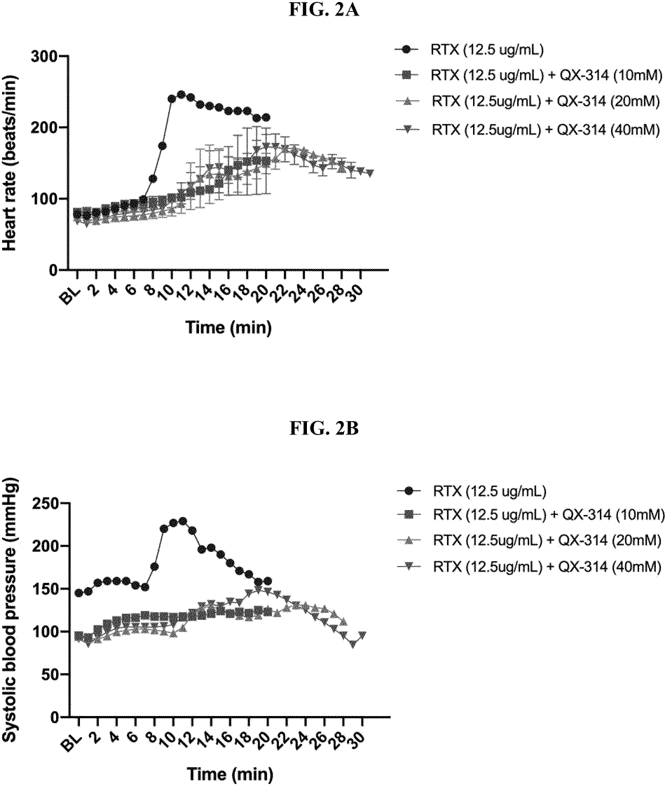

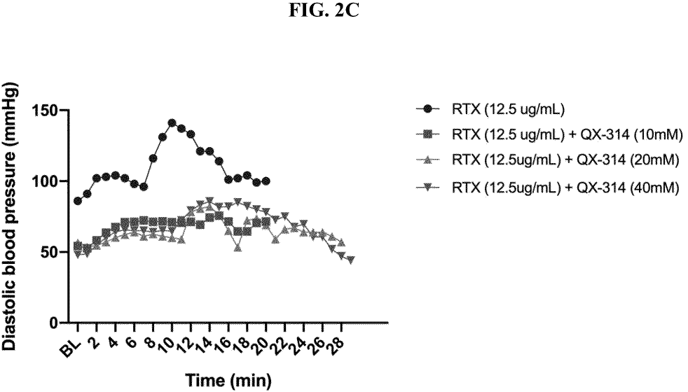

We evaluated the effects of the TRPV1 modulator RTX alone and in combination with QX-314 on cardiovascular function, specifically heart rate and blood pressure, in pigs. Animals were first sedated with telazol (4-8 mg/kg, intramuscular), intubated, and mechanically ventilated. General anesthesia was maintained with isoflurane (1-2%, inhaled). A 12-lead surface electrocardiogram and arterial blood pressure were continuously obtained using a GE Healthcare CardioLab system. Local anesthesia with 1% lidocaine was then administered to the left sternocostal angle before making a small incision. Percutaneous access into the pericardial space was obtained using a Touhy needle under fluoroscopic guidance (see FIG. 1). Contrast was used to visualize the advancement of the needle tip, and intrapericardial access was confirmed by passing a wire through the needle and inducing cardiac ectopy. After confirming access, a sheath was introduced into the pericardial space over the wire. A 12.5 µg/mL RTX solution was prepared by first creating a stock solution of 0.1 mg/mL by dissolving 0.5 mg of RTX powder in 2.5 mL of dimethyl sulfoxide and 2.5 mL of 0.9% sodium chloride, and then diluting the stock solution to 12.5 µg/mL by adding 1.87 mL of it to 13.13 mL of 0.9% sodium chloride. To prepare a RTX solution in combination with QX-314, 50, 100, 150, or 200 mg of QX-314 was added to create a 12.5 µg/mL RTX solution with 10, 20, 30, or 40 µM of QX-314, respectively. In one subset of animals (control group), 15 mL of a RTX solution alone was intrapericardially administered and completely aspirated 20 minutes later. In another subset of animals (treatment group), 15 ml of a RTX solution in combination with QX-314 was intrapericardially administered and completely aspirated 20 minutes later. We tested RTX at a concentration of 12.5 µg/mL alone and in combination with QX-314 at concentrations of 10, 20, 30, and 40 µM to determine concentrations of RTX and QX-314 in the mixture that would result in desensitization of TRVP1 neurons without significant sympathetic activation. With the administration of RTX alone, there was a significant increase in heart rate (FIG. 2A) and blood pressure (FIG. 2B, FIG. 2C) from baseline. However, administration of RTX in combination with QX-314 attenuated the increase in heart rate and blood pressure (FIGS. 2A-C).

To evaluate whether the TRPV1 neurons had been desensitized following administration of the mixture of RTX and QX-314, the TRPV1 agonist capsaicin was subsequently administered to the animals in the RTX in combination with 20 µM QX-314 group. Four weeks after intrapericardial administration of RTX in combination with 20 µM QX-314, animals were again sedated, intubated, and mechanically ventilated. A midline sternotomy was performed, and the pericardium was opened to expose the heart. A 20 µg/mL solution of capsaicin was applied to the basal anterior surface of the heart. There was minimal change in the heart rate and blood pressure over 30 minutes following the administration of capsaicin, indicating that the TRPV1 neurons had indeed been desensitized by the administration of RTX while sympathetic activation was blunted by QX-314. We expect this minimal change in the heart rate and blood pressure upon administration with a subsequent TRPV1 modulator would be seen for a period of 30 minutes to 6 months following the initial administration of the TRPV1 modulator with QX-314.

We expect to find that the concentration of RTX of in the range of 0.1 µg/mL to 125 µg/mL and the concentration of QX-314 of 10 mM to 40 mM to result in desensitization of TRPV1 neurons without sympathetic activation. We expect that these results would be obtained using TRPV1 modulators other than RTX in combination with QX-314, including but not limited to other compounds such as capsaicin, nordihydrocapsaicin, dihydrocapsaicin, homodihydrocapsaicin, homocapsaicin, nonivamide, bradykinin, RTX, Iodo-RTX, tinyatoxin, allyl isothiocyanate, N- arachidonoylaminophenol, N-Vanillylarachidonamide, N-oleoyl-dopamine, olvanil, palvanil, cannabidiol, capsiate, capsaicin O-ethyl (trimethylammonium) acetate, capsaicin O-butyl(trimethylammonium) acetate, capsaicin O-tetraethylammonium acetate, or zucapsaicin, as well as changes in temperature or pH that modulate TRPV1 activity.

1. A method of co-administering QX-314 and a TRPV1 modulator to prevent sympathetic activation resulting from administration of a TRPV1 modulator alone comprising administering QX-314 and the TRPV1 modulator to a subject in need thereof.

2. The method of claim 1, wherein QX-314 is administered before the TRPV1 modulator.

3. The method of claim 1, wherein QX-314 and the TRPV1 modulator are administered at the same time.

4. The method of claim 1, wherein QX-314 is administered after the TRPV1 modulator.

5. The method of claims 1-4, wherein administering QX-314 comprises administration by at least one of topical, subcutaneous, epicardial, epidural, intrathecal, peri or intra-ganglionic, vascular, intraarticular, interarticular, pericardial, intrapericardial, or intravesical administration.

6. The method of claims 1-4, The method of claim 1, wherein administering the TRPV1 modulator comprises administration by at least one of topical, subcutaneous, epicardial, epidural, intrathecal, peri or intra-ganglionic, vascular, intraarticular, interarticular, pericardial, intrapericardial, or intravesical administration.

7. The method of claim 1,wherein administering QX-314 and the TRPV1 modulator comprises administration by two different methods of administration.

8. The method of claims 1-6, wherein administering QX-314 and the TRPV modulator comprises administration by the same method of administration.

9. The method of claim 1, wherein the TRPV1 modulator is selected from the group consisting of one or more of the following:
capsaicin, nordihydrocapsaicin, dihydrocapsaicin, homodihydrocapsaicin, homocapsaicin, nonivamide, bradykinin, RTX, Iodo-RTX, tinyatoxin, allyl isothiocyanate, N- arachidonoylaminophenol, N-Vanillylarachidonamide, N-oleoyl-dopamine, olvanil, palvanil, cannabidiol, capsiate, capsaicin O-ethyl (trimethylammonium) acetate, capsaicin O-butyl(trimethylammonium) acetate, capsaicin O-tetraethylammonium acetate, zucapsaicin, or change in temperature or pH.

10. The method of claim 1, wherein the step of administering QX-314 and the TRPV1 modulator to a subject in need thereof comprises administering QX-314 of a concentration of about 1 mM to about 100 mM to the subject.

11. The method of claim 1, wherein the step of administering QX-314 and the TRPV1 modulator to a subject in need thereof comprises administering the TRPV1 modulator of a concentration of about 0.1 µg/ml to about 125 µg/ml to the subject.

12. The method ofclaim 1, wherein the step of administering QX-314 and the TRPV1 modulator to a subject in need thereof results in preventing sympathetic activation for about 0 to about 60 minutes following the step of administering QX-314 and the TRPV1 modulator that would result if the subject had been administered with TRPV1 modulator alone.

13. A composition to prevent sympathetic activation comprising QX-314 and a TRPV1 modulator.

14. The composition of claim 13, wherein the composition comprises a mixture of QX-314 and the TRPV1 modulator.

15. The composition of claim 13, wherein the TRPV1 modulator is selected from the group consisting of one or more of the following: capsaicin, nordihydrocapsaicin, dihydrocapsaicin, homodihydrocapsaicin, homocapsaicin, nonivamide, bradykinin, RTX, Iodo-RTX, tinyatoxin, allyl isothiocyanate, N- arachidonoylaminophenol, N-Vanillylarachidonamide, N-oleoyldopamine, olvanil, palvanil, cannabidiol, capsiate, capsaicin O-ethyl (trimethylammonium) acetate, capsaicin O-butyl(trimethylammonium) acetate, capsaicin O-tetraethylammonium acetate, or zucapsaicin, or a compound that changes temperature or pH.

16. The composition of claim 13, wherein QX-314 comprises a concentration of about 1 mM to about 100 mM.

17. The composition of claim 13, wherein the TRPV1 modulator comprises a concentration of about 0.1 µg/ml to about 125 µg/ml.

18. The composition of claim 13, wherein the composition further comprises an excipient.

19. The composition of claim 18, wherein the excipient is selected from at least one of the group consisting of ethanol, methanol, polyethylene glycol, dimethyl sulfoxide, sodium chloride, or a cyclodextran.

20-30. (canceled)
